(19) United States
(12) Patent Application Publication
(10) Pub. No.: US 2005/0025710 A1
(43) Pub. Date: Feb. 3, 2005

(54) Reconstitutable formulation and aqueous suspension of gas-filled microvesicles for diagnostic imaging

(76) Inventors: Michel Schneider, Troinex (CH); Yan Feng, Grand-Lancy (CH); Jean Brochot, Feigeres (FR); David Lazurus, Saint-Julien-en-Genevois (FR)

Correspondence Address: Bracco Research USA, 305 College Road East, Princeton, NJ, 08540, US

(21) Appl. No.: 10/630,375

(22) Filed: Jul. 29, 2003

(51) Int. Cl. A61K 49/00

(52) U.S. Cl. 424/9.52

(57) ABSTRACT

The invention relates to a formulation comprising a dry material (e.g. lyophilised or spray dried) comprising at least one film forming surfactant and a gas or a gas mixture usable in diagnostic imaging, to a process for the preparation thereof and to a suspension obtainable by reconstituting said formulation with an aqueous carrier for use in contrast imaging. The invention also relates to a container comprising the dry material in contact with a gas. The gas associated with the dry material is at a pressure lower than the atmospheric pressure.

### TECHNICAL FIELD

The present invention relates to a process for the preparation of dry or lyophilized formulations useful for preparing gas containing contrast agents usable in diagnostic imaging.

The invention also includes dry formulations prepared by this process, which may be reconstituted to form contrast agent suspensions useful in diagnostic imaging. The invention further includes suspensions of gas filled microbubbles useful in diagnostic imaging prepared using dry formulations of the invention as well as containers or two component kits containing the dry formulations of the invention.

### BACKGROUND OF INVENTION

Rapid development of ultrasound contrast agents in the recent years has generated a number of different formulations, which are useful in ultrasound imaging of organs and tissue of human or animal body. These agents are designed to be used primarily as intravenous or intra-arterial injectables in conjunction with the use of medical echographic equipment which employs for example, B-mode image formation (based on the spatial distribution of backscatter tissue properties) or Doppler signal processing (based on Continuous Wave or pulsed Doppler processing of ultrasonic echoes to determine blood or liquid flow parameters). Injectable formulations useful as ultrasound contrast agents, which comprise suspensions of gas microbubbles in aqueous liquid carriers may basically be divided into two categories. Free gas bubbles are not included in these categories since they are not stable enough to be useful as ultrasound contrast agents. Interest has accordingly been shown in methods of stabilising gas bubbles for echography and other ultrasonic studies, for example using emulsifiers, oils, thickeners or sugars, or by entraining or encapsulating the gas or a precursor therefore in a variety of systems. These stabilized gas bubbles are generally referred to in the art as gas-filled “microvesicles” or “microspheres”, and may be divided into two main categories. A first category of stabilized bubbles is generally referred to in the art as “microbubbles” and includes aqueous suspensions in which the bubbles of gas are bounded at the gas/liquid interface by a very thin envelope involving a surfactant (i.e. an amphiphilic material) bound at the gas to liquid interface. A second category of microvesicles is generally referred to in the art as “microballoons” or “microcapsules” and includes suspensions in which the bubbles of gas are surrounded by a solid material envelope formed of natural or synthetic polymers. A further kind of ultrasound contrast agent comprises suspensions of porous microparticles of polymers or other solid material carrying gas microbubbles entrapped within the pores of the microparticles. More on these different types of formulations may be found, for instance in U.S. Pat. No. 4,466,442, EP 0 123 235, EP 0 324 938, U.S. Pat. No. 5,271,928, U.S. Pat. No. 5,711,933, U.S. Pat. No. 4,900,540, U.S. Pat. No. 5,230,882, incorporated by reference herein in their entirety.

Use of suspensions of gas-filled microvesicles in carrier liquid, as efficient ultrasound reflectors is well known in the art. The development of microbubble suspensions as echopharmaceuticals for enhancement of ultrasound imaging followed early observations that rapid intravenous injections of aqueous solutions can cause dissolved gases to come out of solution by forming bubbles. Due to their substantial difference in acoustic impedance relative to blood, these intravascular gas bubbles were found to be excellent reflectors of ultrasound. Injection of aqueous suspensions of gas microbubbles into the blood stream of a living organism strongly reinforces ultrasonic echography imaging, thus enhancing the visualisation of internal organs. Since imaging of organs and deep seated tissues can be crucial in establishing medical diagnosis, a lot of effort has been devoted to the development of stable suspensions of highly concentrated gas microbubbles which at the same time would be simple to prepare and administer, would contain a minimum of inactive species and would be capable of long storage and simple distribution.

One of the problems still open now is the fact that the reproducibility and analysis of echographic tests may be fairly difficult. In addition, some techniques produce bubbles in a wide range of diameters (up to 50 μm) which prevents their use as echographic agents in certain applications since echography of the left heart and of the general circulation requires bubbles sizes smaller than 8-10 μm. Microbubbles formulations whose distribution and storage would not present problems are particularly important.

Easy-to-produce aqueous suspensions usable as imaging agents in ultrasonic echography are disclosed for example in U.S. Pat. No. 5,271,928 (from WO 91//15244), U.S. Pat. No. 5,445,813, U.S. Pat. No. 5,413,774, U.S. Pat. Nos. 5,556,610, 5,597,549 and co-pending European patent application EP 03002375.8 (filed on 4^{th} Feb. 2003), which are here incorporated by reference in their entirety.

Storage and transportation of such contrast agents is made significantly more efficient if the microbubbles can be stored in a dried form. The dry form does not require the need of temperature control and special storage facilities for storage and transportation. Thus production of lyophilised formulations, which may be stably and reproducibly regenerated to yield echogenic contrast agents is particularly desirable.

One of the purposes of pharmaceutical packaging is to provide adequate product protection. The container forms the primary barrier required for assurance of quality over the shelf life of the product. Certain products, particularly those that are lyophilised, must also be protected from moisture and may need to be protected from oxygen. Contrast agents comprising gas filled microbubbles are particularly storage sensitive. The problems of storage are intrinsic to aqueous gas suspensions, which due to their very nature may undergo phase separation or segregation, gas bubble coalescence, gas diffusion and, after long periods, even precipitation of various additives.

In addition, convenient containers have to be selected. Storing lyophilised contrast agents at atmospheric pressure also requires very adapted connection systems as well as containers. Reconstitution of lyophilised product with a given volume of aqueous liquid carrier will create an overpressure inside the container. Convenient connection systems or adapters may be for example those disclosed in WO 98/13006 as well as in U.S. Pat. No. 4,787,898, incorporated by reference herein in their entirety. The above adapters require the presence of a vent in their structure in order to adjust the container's pressure after reconstitution of the contrast agent. The cited devices for sealing closed containers and adapters are expensive and are not very easy to handle.

Recently it has been disclosed that pharmaceuticals may also be sealed in containers under reduced pressure. Daukas et al. in PDA Journal of Pharmaceutical Science & Technology Vol. 53(1), pp.31-39 (1999) describes comparative methods for integrity testing of vials sealed at reduced pressures for lyophiles. An advantage of sealing at reduced pressure may lie in promoting ease of reconstitution as the vacuum draws diluent into the vial. Buckley in PDA Journal of Pharmaceutical Science & Technology Vol. 48(4), pp.189-196 (1994) presented the time dependence of pressure in lyophilisation vials. Vials containing lyophilised products are frequently sealed at a nominal chamber pressure Pc<50 mtorr (1 torr=1 mm Hg=1.333 mbar), however vial pressures may also be encountered in the range of 2-100 torr. Controlling the storage atmosphere and facilitating dissolution of solid products before use may also represent other advantages of containers sealed under reduced pressure.

Another problem to be solved is to prevent the alteration of the acoustic properties (echogenic response) after the reconstitution in an aqueous suspension of a dry powder (and particularly a lipid-containing dried powder) stored for a long time under reduced pressure. In addition, one of the problems of reconstituted lyophilised microbubbles lies in the possible presence of very large bubbles (>10 μm). In order to avoid the presence of big bubbles, one aspect of the present invention relates to the control of the size of the bubbles after reconstitution.

### SUMMARY OF INVENTION

The invention broadly relates to a process for the preparation of a dry formulation which upon reconstitution yields a gas or gas mixture containing-contrast agent usable in diagnostic imaging. The invention also relates to a dry formulation obtained by this process as well as to a container or a two-component kit containing said formulation and said gas, for the reconstitution of an injectable suspension of gas filled microbubbles and their use as contrast agents in diagnostic imaging of human and animal body. The dry formulation is preferably a lyophilized formulation.

According to an aspect of the invention, a compostion is obtained comprising a gas under reduced pressure in contact with a dry formulation which comprises one or more film-forming surfactants useful for preparing contrast agents which, upon dissolution in water or in an aqueous carrier liquid, will form an injectable aqueous suspension of gas filled microbubbles usable as imaging contrast agent in diagnostic imaging. According to the invention, it is possible to substantially prevent or significantly reduce the alteration of the acoustic properties (echogenic response) during storage. By adjusting the range of the reduced pressures applied to the container, the acoustic properties will not be affected by storage of the lyophilised powder for extended periods. Thus, one advantage of the present invention is to prevent the alteration of the acoustic properties after the reconstitution in an aqueous suspension of a lyophilised material which has been stored in a container under reduced pressure at a temperature of 20° C. or at 30° C. or even at 40° C. during a period of one month or even two months or more.

According to another aspect, the invention relates to a method for preparing an injectable reconstituted suspension of gas filled microbubbles usable as contrast agent in diagnostic imaging according to claim 1. In particular, the above composition of reduced pressure gas and dry material is admixed with water or a physiologically acceptable aqueous carrier liquid.

According to yet another aspect, the invention relates to the use of the above composition of reduced pressure gas and lyophilised material in a physiologically or pharmaceutically acceptable aqueous liquid carrier for the manufacture of an injectable aqueous suspension of gas filled microbubbles for use in diagnostic imaging.

According to a further aspect, the invention relates to a method of diagnostic imaging which comprises administering to a subject a contrast-enhancing amount of the above contrast agent and imaging at least a part of said subject by diagnostic imaging. According to this method, said subject is a vertebrate and said contrast agent is introduced into the vasculature or into a body cavity of said vertebrate.

According to yet a further aspect, the invention relates to a container containing the above composition of reduced pressure gas and lyophilised material. Still in a further aspect the invention relates to a two component kit comprising, as the first component, a container comprising a composition as defined above and as the second component a physiologically acceptable carrier liquid which, when admixed with the first component, provides an injectable contrast agent.

As used herein, the terms physiologically acceptable or biocompatible refer to any compound or composition which can be administered to a patient without negatively affecting or substantially altering the normal functioning (physiology) of the organism of said patient.

### BRIEF DESCRIPTION OF THE DRAWINGS

FIG. 1 is an example of a suitable container according to the invention.

FIG. 2 is a graph representing the variation in number of the bubbles concentration upon storage at 25° C.

FIG. 3 is a graph representing the variation of the resistance to pressure of the bubbles upon storage at 25° C.

FIG. 4 is a graph representing the variation in number of the bubbles concentration upon storage at 40° C.

FIG. 5 is a graph representing the variation of the resistance to pressure of the bubbles upon storage at 40° C.

### DETAILED DESCRIPTION OF THE INVENTION

The present invention relates, inter alia to a novel composition which, upon being contacted with a physiologically acceptable liquid aqueous carrier, forms a suspension of gas-filled microvesicles stabilized by a layer of film-forming material, which is suitable for use as contrast agent in diagnostic imaging.

In particular, said composition, which is preferably contained into a gas-sealed container, comprises, as a first component, a material in the form of a dry powder and comprising at least one film-forming surfactant and, as a second component, a gas in contact with said dry material, said gas having a pressure lower than atmospheric pressure.

A suitable process for preparing said composition comprises:
a) preparing a dry material comprising at least one film-forming surfactant into a container;
b) applying a vacuum to said container;
c) introducing a gas into said container, up to a pressure which is lower than atmospheric pressure;
d) effecting a sealing closure of said container, thus obtaining the dry material in contact with an atmosphere of said gas having a pressure lower than atmospheric pressure.

Preferably, the preparation of the dry material comprises lyophilising a lyophilisable suspension of said material, to obtain a dried powdered material

The pressure of said gas or gas mixture introduced into said container is preferably from about 100 to about 900 mbar.

More preferably, the pressure of said gas or gas mixture introduced into said container comprises between about 300 and about 700 mbar.

Even more preferably, the pressure of said gas or gas mixture introduced into said container is from about 400 mbar to 600 mbar, in particular of about 500 mbar.

Whenever used in the present description and claims, the term “reduced pressure” includes within its meaning any value of pressure which is lower than atmospheric pressure, i.e. typically lower than 1000 mbar at sea level. As mentioned before, the reduced pressure gas is preferably introduced into the container containing the dry material after a vacuum has been applied in the inside said container. Said vacuum is of at least about 50 mbar or lower, preferably of at least about 0.2 or lower, more preferably of at least about 0.05 mbar or lower, down to e.g. about 0.001 mbar, preferably down to about 0.01 mbar. Typically, the applied vacuum is from about 0.5 mbar to about 0.05 mbar.

In case a relatively low vacuum is applied to the container, e.g. when the pressure in the container is reduced down to about 10-50 mbar, the residual pressure and kind of gas remaining in the container shall be taken into account when introducing the reduced pressure gas into the container, for a correct estimate of the final pressure and composition of the gas contained in the container.

In an alternative embodiment of the invention, the gas is introduced at atmospheric pressure into the container and the reduced pressure is applied just before reconstitution of the contrast agent with a physiologically acceptable liquid carrier. The reduced pressure may be obtained for instance by applying a slight vacuum to said container, down to the desired reduced pressure. In the latter embodiment, the lyophilised contrast agent may be stored in a container under atmospheric pressure prior to application of the vacuum.

With the present invention it is possible to extend the storage life of a dried (e.g. lyophilized) contrast agent and allow reconstitution of stable contrast agents with a reduced percentage of large bubbles. In the present invention, the size of the microbubbles generated is consistently reproducible and in practice is independent of the amount of agitational energy applied. In particular, by using the method of the invention it is possible to substantially decrease the amount of bubbles larger than 10 μm. For instance, more than 50% of large size bubbles (>8 μm) can be eliminated by using a reduced pressure of 500 mbar, as compared with a sample prepared by using atmospheric gas pressure (1000 mbar).

It has been surprisingly found that when the lyophilised or dried material is stored in a container containing a gas under reduced pressure, it shows improved stability after storage for a long period of time (such as, for example, more than one year).

Another advantage of the present invention is that the pressure resistance of the bubbles (the definition of pressure resistance, for example, can be found in EP 0 554 213 incorporated herein by reference in its entirety) obtained after the reconstitution of the lyophilised material in an aqueous suspension is retained even after the lyophilised material has been stored in a container under reduced pressure of a gas or gas mixture at a temperature ranging from 20° C. to 40° C. during a period ranging from one month to six months or even more.

Thus, the stable lyophilised precursor of the contrast agents of the present invention may be stored and transported without need of temperature control of its environment and in particular may be supplied to hospitals and physicians for on site formulation into a ready to-use administrable suspension without requiring such users to have special storage facilities.

Indeed, lyophilized products according to the invention have proven to be storage stable for several months under ambient conditions.

The microbubble dispersions or suspensions generated upon reconstitution in water (or other reconstitution liquids) may be stable for a considerable period time, e.g. up to at least 12 hours.

A further advantage of the present invention is that, because of the dried contrast agent being sealed at reduced pressure, it can be easier reconstituted as the introduction of the diluent into the vial is facilitated by the reduced pressure Sealing under reduced pressure also permits the storage atmosphere to be controlled and facilitates dissolution of lyophilised solid products before use.

An advantage of the present invention is that because the gas or gas mixture is introduced into the vial containing the lyophilised product under reduced pressure, less gas must be used. This is a significant advantage because many of the preferred gases such as, for example, perfluorocarbons or hyperpolarized gases are very expensive.

Indeed, applying a reduced pressure in the lyophilisation chamber may reduce the amount of gas from 50 to 80%.

For example, using a pressure of 200 mbar allows an economy of 80% of the gas quantity as compared to a contrast agent prepared at atmospheric pressure.

This results also in an ecological improvement since less perfluorocarbon gases are released in the atmosphere.

Moreover, in the case of very expensive gases such as the hyperpolarized helium, xenon or neon used for magnetic resonance imaging (MRI), the saving may be significant.

Diagnostic imaging means in the present invention any modality able to visualize a particular region of a human or non-human body. For example the following modalities can be included: ultrasonic echography, magnetic resonance imaging (MRI), X-ray radiography and scintigraphy.

Biocompatible Gas

In the present invention, the reconstituted contrast agent may contain a gas or gas precursor (eg. a compound or mixture of compounds which are substantially in gaseous form (including vapour) at normal human body temperatures (37° C.) (such as, for instance, C₅F₁₂, C₆F₁₄).

Any biocompatible gas, gas precursor or mixture may be employed. The gas will be selected depending on the chosen modality.

The gas may thus comprise, for example: air; nitrogen; oxygen; carbon dioxide; hydrogen; nitrous oxide; a noble or inert gas such as helium, argon, xenon or krypton; a radioactive gas such as Xe¹³³ or Kr⁸¹; a hyperpolarized noble gas such as hyperpolarized helium or hyperpolarized xenon or hyperpolarized neon; a low molecular weight hydrocarbon (e.g. containing up to 7 carbon atoms), for example an alkane such as methane, ethane, a propane, a butane or a pentane, a cycloalkane such as cyclobutane or cyclopentane, an alkene such as propene or a butene, or an alkyne such as acetylene; an ether; a ketone; an ester; halogenated gases, preferably fluorinated gases, such as or halogenated low molecular weight hydrocarbons (e.g. containing up to 7 carbon atoms); or a mixture of any of the foregoing. Where a halogenated hydrocarbon is used, preferably at least some of the halogen atoms in halogenated gases advantageously are fluorine atoms. Preferred halogenated hydrocarbons are perfluorocarbons, i.e. an hydrocarbons where all the hydrogen atoms are replaced by fluorine atoms (perfluorinated hydrocarbons)

Examples of halogenated gases include sulfur hexafluoride; fluorinated, preferably perfluorinated, ketones such as perfluoroacetone; and fluorinated, preferably perfluorinated, ethers such as perfluorodiethyl ether.

Fluorinated gases are preferred, in particular perfluorinated gases, especially for use in ultrasound contrast agents. Fluorinated gases include materials which contain at least one fluorine atom such as SF₆, fluorinated hydrocarbons (organic compounds containing one or more carbon atoms and fluorine) and perfluorocarbons.

As mentioned before, the term perfluorocarbon refers to compounds containing only carbon and fluorine atoms and includes saturated, unsaturated, and cyclic perfluorocarbons. Examples of of biocompatible, physiologically acceptable perfluorocarbons are: perfluoroalkanes, such as perfluoromethane, perfluoroethane, perfluoropropanes, perfluorobutanes (e.g. perfluoro-n-butane, optionally in admixture with other isomers such as perfluoro-isobutane), perfluoropentanes, perfluorohexanes or perfluoroheptanes; perfluoroalkenes, such as perfluoropropene, perfluorobutenes (e.g. perfluorobut-2ene) or perfluorobutadiene; perfluoroalkynes (e.g. perfluorobut-2-yne); and perfluorocycloalkanes (e.g. perfluorocyclobutane, perfluoromethylcyclobutane, perfluorodimethylcyclobutanes, perfluorotrimethylcyclobutanes, perfluorocyclopentane, perfluoromethylcyclopentane, perfluorodimethylcyclopentanes, perfluorocyclohexane, perfluoromethylcyclohexane and perfluorocycloheptane). Preferred saturated perfluorocarbons have the formula CₙFₙ₊₂, where n is from 1 to 12, preferably from 2 to 10, most preferably from 3 to 8 and even more preferably from 3 to 6. Suitable perfluorocarbons include, for example, CF₄, C₂F₆, C₃F₈, C₄F₈, C₄F₁₀ C₅F₁₂, C₆F₁₂, C₇F₁₄, C₈F₁₈, and C₉F₂₀.

Particularly preferred gases are SF₆ or perfluorocarbons selected from the group consisting of CF₄, C₂F₆, C₃F₈, C₄F₈ and C₄F₁₀ or mixtures thereof, SF₆, C₃F₈ or C₄F₁₀ being particularly preferred.

As mentioned above the gas can be a mixture of gases, selected among those disclosed above. In particular the following combinations are particularly preferred: a mixture of gases (A) and (B) in which the gas (B) is a fluorinated gas, preferably selected from SF₆, CF₄, C₂F₆, C₂F₈, C₃F₆, C₃F₈, C₄F₆, C₄F₈, C₄F₁₀, C₅F₁₀, C₅F₁₂ or mixtures thereof, and (A) is selected from air, oxygen, nitrogen, carbon dioxide or mixtures thereof. Preferably, the amount of gas (B) is from 0.5% to 41% by vol.

In certain circumstances it may be desirable to include a precursor to a gaseous substance (e.g. a material that is capable of being converted to a gas in vivo). Preferably the gaseous precursor and the gas it produces are physiologically acceptable. The gaseous precursor may be pH-activated, photo-activated, temperature activated, etc. For example, certain perfluorocarbons may be used as temperature activated gaseous precursors. These perfluorocarbons, such as perfluoropentane, have a liquid/gas phase transition temperature above room temperature (or the temperature at which the agents are produced and/or stored) but below body temperature; thus, they undergo a phase shift and are converted to a gas within the human body.

For ultrasonic echography, the biocompatible gas or gas mixture is preferably selected from the group consisting of air, nitrogen, carbon dioxide, helium, krypton, xenon, argon, methane, hyperpolarized gases, halogenated hydrocarbons (including fluorinated gases such as perfluorocarbons and sulfur hexafluoride and mixtures thereof. Advantageously, perfluorocarbons (in particular C₄F₁₀ or C₃F₈) or SF₆ can be used, optionally in admixture with air or nitrogen.

The hyperpolarized gases may preferably be selected from hyperpolarized helium or hyperpolarized xenon or hyperpolarized neon and mixtures thereof.

Gas precursors such as perfluorocarbons with a boiling point close to room temperature (e.g. C₅F₁₂ or C₆F₁₄) may also be used in the present invention. One of the advantages of the reduced pressure is in fact to sustain the gas or gas precursor in its vapor state and thus to avoid or substantially reduce the condensation of said gas precursor.

Particularly preferred contrast agents usable in MRI will contain hyperpolarized noble gases such as hyperpolarized neon, hyperpolarized helium, hyperpolarized xenon, or mixtures thereof with air, CO₂, oxygen, nitrogen, helium, or all the halogenated hydrocarbons as defined above.

For scintigraphy, contrast agents according to the invention will preferably contain radioactive gases such as Xe¹³³ or Kr⁸¹ or mixtures thereof with air, CO₂, oxygen, nitrogen, helium, or all the halogenated hydrocarbons as defined above.

Biocompatible Surfactants

Film-forming surfactants conveniently employed in this invention are selected from amphipathic compounds capable of forming a stabilizing layer of material around the gas bubbles dispersed in the aqueous carrier. The amphipathic compound can be a synthetic or naturally-occurring biocompatible compound and may include, for example a film forming lipid, preferably a phospholipid. Examples of amphipathic compounds include, for instance fatty acids; lysolipids; phospholipids; fatty acids such as palmitic acid, stearic acid, arachidonic acid or oleic acid; lipids bearing polymers, such as chitin, hyaluronic acid, polyvinylpirrolidone or polyethylene glycol (PEG), also referred as “pegylated lipids”, with preferred lipids bearing polymers including PEG-modified phospholipids; lipids bearing sulfonated mono- di-, oligo- or polysaccharides; cholesterol, cholesterol sulfate or cholesterol hemisuccinate; tocopherol hemisuccinate; lipids with ether or ester-linked fatty acids; polymerized lipids (a wide variety of which are well known in the art); diacetyl phosphate; dicetyl phosphate; stearylamine; ceramides; polyoxyethylene fatty acid esters, polyoxyethylene fatty alcohols, polyoxyethylene fatty alcohol ethers, polyoxyethylated sorbitan fatty acid esters, glycerol polyethylene glycol ricinoleate, ethoxylated soybean sterols, ethoxylated castor oil, polyoxyethylene-polyoxypropilene polymers, or polyoxyethylene fatty acid stearates; sterol aliphatic acid esters including cholesterol sulfate, cholesterol butyrate, cholesterol iso-butyrate, cholesterol palmitate, cholesterol stearate, lanosterol acetate, ergosterol palmitate, or phytosterol n-butyrate; sterol esters of sugar acids including cholesterol glucoronides, lanosterol glucoronides, 7-dehydrocholesterol glucoronide, ergosterol glucoronide, cholesterol gluconate, lanosterol gluconate, or ergosterol gluconate; esters of sugar acids and alcohols including lauryl glucoronide, stearoyl glucoronide, myristoyl glucoronide, lauryl gluconate, myristoyl gluconate, or stearoyl gluconate; esters of sugars with aliphatic acids including sucrose laurate, fructose laurate, sucrose palmitate, sucrose stearate, glucuronic acid, gluconic acid or polyuronic acid; saponins including sarsasapogenin, smilagenin, hederagenin, oleanolic acid, or digitoxigenin; glycerol dilaurate, glycerol trilaurate, glycerol dipalmitate, glycerol or glycerol esters including glycerol tripalmitate, glycerol distearate, glycerol tristearate, glycerol dimyristate, glycerol trimyristate; long chain alcohols including n-decyl alcohol, lauryl alcohol, myristyl alcohol, cetyl alcohol, or n-octadecyl alcohol; 6-(5-cholesten-3β-yloxy)-1-thio-β-D-galactopyranoside; digalactosyl-diglyceride; 6-(5-cholesten-3β-yloxy)hexyl-6-amino-6-deoxy-1-thio-β-D-galacto-pyranoside; 6-(5-cholesten-3β-yloxy)hexyl-6-amino-6-deoxyl-1-thio-β-D-manno-pyranoside; 12-(((7′-diethylaminocoumarin-3-yl)carbonyl)methylamino)octade-canoic acid; N-[12-(((7′-diethylaminocoumarin-3-yl)carbonyl)methylamino)-octadecanoyl]-2-aminopalmitic acid; N-succinyldioleylphosphatidylethanolamine; 1,2-dioleyl-sn-glycerol; 1,2-dipalmitoyl-sn-3-succinylglycerol; 1,3-dipalmitoyl-2-succinylglycerol; 1-hexadecyl-2-palmitoylglycerophosphoethanolamine or palmitoylhomocysteine; and mixtures or combinations thereof.

For forming an effective stabilizing film, at least one of the compounds forming the microvesicles' envelope shall preferably be a phospholipid, optionally in admixture with any of the other above cited surfactants. Examples of suitable phospholipids include esters of glycerol with one or two (equal or different) molecules of fatty acids and with phosphoric acid, wherein the phosphoric acid residue is in turn bonded to a hydrophilic group, such as choline (phosphatidylcholines—PC), serine (phosphatidylserines—PS), glycerol (phosphatidylglycerols—PG), ethanolamine phosphatidylethanolamines (PE), inositol (phosphatidylinositol), and the like groups. Fatty acids present in the phospholipids are in general long chain aliphatic acids, typically containing from 12 to 24 carbon atoms, preferably from 14 to 22, that may be saturated or may contain one or more unsaturations. Examples of suitable fatty acids are lauric acid, myristic acid, palmitic acid, stearic acid, arachidic acid, behenic acid, oleic acid, linoleic acid, and linolenic acid. Mono esters of phospholipid are also known in the art as the “lyso” forms of the phospholipids.

Further examples of phospholipid are phosphatidic acids, i.e. the diesters of glycerol-phosphoric acid with fatty acids, sphingolipids such as sphingomyelins, i.e. those phosphatidylcholine analogs where the residue of glycerol diester with fatty acids is replaced by a ceramide chain, cardiolipins, i.e. the esters of 1,3-diphosphatidylglycerol with a fatty acid, glycolipids such as gangliosides GM1 or GM2 or cerebrosides; glucolipids; sulfatides and glycosphingolipids.

As used herein, the term phospholipids include either naturally occurring, semisynthetic or synthetically prepared products that can be employed either singularly or as mixtures.

Examples of naturally occurring phospholipids are natural lecithins (phosphatidylcholine (PC) derivatives) such as, typically, soya bean or egg yolk lecithins.

Examples of semisynthetic phospholipids are the partially or fully hydrogenated derivatives of the naturally occurring lecithins.

The term phospholipid further includes modified phospholipid, e.g. phospholipids where the hydrophilic group is in turn bound to another hydrophilic group. Examples of modified phospholipids are PEG (polyethylenglycol) modified phospholipids, such as PEG-modified ethylenaminephospholipids, where the hydrophilic moiety of the phospholipid (e.g. ethanolamine) is linked to a PEG molecule of variable molecular weight (from 300 to 5000 daltons), such as DPPE-PEG, i.e. DPPE having a PEG polymer attached thereto, including, for example, DPPE-PEG2000, which refers to DPPE having attached thereto a PEG polymer having a mean average molecular weight of about 2000. Preferably the phospholipids are saturated. Preferred examples of phospholipids are for instance dilauryloyl-phosphatidylcholine (DLPC), dimyristoylphosphatidylcholine (DMPC), dipalmitoyl-phosphatidylcholine (DPPC), diarachidoylphosphatidylcholine (DAPC), distearoyl-phosphatidylcholine (DSPC), dioleoylphosphatidylycholine (DOPC), 1,2 Distearoyl-sn-glycero-3-Ethylphosphocholine (Ethyl-DSPC), dipentadecanoyl-phosphatidylcholine (DPDPC), 1-myristoyl-2-palmitoylphosphatidylcholine (MPPC), 1-palmitoyl-2-myristoylphosphatidylcholine (PMPC), 1-palmitoyl-2-stearoylphosphatid-ylcholine (PSPC), 1-stearoyl-2-palmitoyl-phosphatidylcholine (SPPC),), 1-palmitoyl-2-oleylphosphatid-ylcholine (POPC), 1-oleyl-2-palmitoyl-phosphatidylcholine (OPPC), dilauryloyl-phosphatidylglycerol (DLPG) and its alkali metal salts, diarachidoylphosphatidylglycerol (DAPG) and its alkali metal salts, dimyristoylphosphatidylglycerol (DMPG) and its alkali metal salts, dipalmitoyl-phosphatidylglycerol (“DPPG”) and its alkali metal salts, distearolyphosphatidylglycerol (DSPG) and its alkali metal salts, dioleoylphosphatidylglycerol (DOPG) and its alkali metal salts, dimyristoyl phosphatidic acid (DMPA) and its alkali metal salts, dipalmitoyl phosphatidic acid “DPPA) and its alkali metal salts, distearoyl phosphatidic acid (DSPA), diarachidoyl phosphatidic acid (DAPA) and its alkali metal salts, dimyristoyl phosphatidyl-ethanolamine (DMPE), dipalmitoyl phosphatidylethanolamine (DPPE), DPPE-PEG, distearoyl phosphatidyl-ethanolamine (DSPE), DSPE-PEG, dioleylphosphatidylethanolamine (DOPE), diarachidoylphosphatidylethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP) and distearoyl sphingomyelin (DSSP).

Neutral and charged phospholipids can satisfactorily be employed in the process of the present invention, as well as mixtures thereof. In addition, also mixtures of neutral phospholipids with charged lipids, such as 1,2-dipalmitoyl-3-trimethylammonium-propane (DPTAP) or 1,2-stearoyl-3-trimethylammonium-propane (DSTAP) can be used. As used herein and in the prior art, the term “charged” in relation with (phospho)lipids means that the individual (phospho)lipid molecules have an overall net charge, be it positive or, more frequently, negative. Preferably, blends of two or more phospholipids and or lipids, at least one with a neutral charge and at least one with an overall net charge, are employed. More preferably, blends of two or more phospholipids, at least one with neutral and at least one with negative charge are employed. The amount of charged phospholipid, may vary from about 95% to about 5% by weight, with respect to the total amount of phospholipid, preferably from 80% to 20% by weight. The presence of at least minor amounts, such as 5% to 20% by wt. with respect to the total weight of phospholipid, of a (negatively) charged phospholipid may help preventing aggregation of bubbles or emulsion droplets. It is however possible to use a single phospholipid, neutral or charged, or a blend of two or more phospholipids, all neutral or all with an overall net charge.

Particularly preferred phospholipids are DAPC, DPPA, DSPA, DMPS, DPPS, DSPS and Ethyl-DSPC. Most preferred are DSPA, DPPS or DSPS.

Preferred mixtures of phospholipids are mixtures of DPPS with DPPC, DSPC or DAPC (from 95/5 to 5/95 w/w), mixtures of DSPA with DSPC or DAPC (from 95/5 to 5/95 w/w), mixtures or DSPG or DPPG with DSPC or mixtures of DSPC with Ethyl-DSPC. Most preferred are mixtures of DPPS/DSPC (from 50/50 to 10/90 w/w) or DSPA/DSPC (from 50/50 to 20/80 w/w).

The amount of phospholipid is generally comprised between about 0.005 and about 1.0% by weight with respect to the total weight of the final suspension of gas filled microbubbles. Larger amounts might of course be employed but considering that the end product is an injectable contrast agent, it is preferred not to use excess of additives unless strictly necessary to provide for a stable and suitable product. In general, by using an amount of phospholipid larger than that indicated as the upper limit of the above range, essentially no or a very negligible improvement is observed in terms of bubble population, bubble size distribution, and bubble stability.

The phospholipids can conveniently be used in admixture with any of the above listed amphipathic compounds, the amount of phospholipids being preferably of at least 50%, more preferably of at least 70% by weight with respect to the total amount of amphipathic material forming the stabilizing envelope of the microvesicle. Thus, additives like cholesterol and/or other non-phospholipid surfactants can optionally be added to one or more of the foregoing phospholipids in proportions ranging from zero to 50% by weight. Other excipients may be present in the dry formulation or may be added with the aqueous carrier upon reconstitution. These include pH regulators, osmolality adjusters, viscosity enhancers, emulsifiers, bulking agents, etc. and may be used in conventional amounts. For instance compounds like polyoxypropylene glycol and polyoxyethylene glycol as well as copolymers thereof, ergosterol, phytosterol, sitosterol, lanosterol, tocopherol, propyl gallate, ascorbyl palmitate, fatty acids such as myristic acid, palmitic acid, stearic acid, arachidic acid or their derivatives and butylated hydroxytoluene. Particularly preferred is palmitic acid. Examples of viscosity enhancers or stabilizers are compounds selected from linear and cross-linked poly- and oligo-saccharides, sugars, hydrophilic polymers like polyethylene glycol.

Since preparation of the dry material typically involves a freeze drying or spray drying step, it may be advantageous to include one or more agents with cryoprotective and/or lyoprotective effect and/or one or more bulking agents, for example an amino-acid such as glycine; a carbohydrate, e.g. a sugar such as sucrose, mannitol, trehalose, glucose, lactose or a cyclodextrin, or a polysaccharide such as dextran; or a polyglycol such as polyethylene glycol. A substantial list of agents with cryoprotective and/or lyoprotective effects is given in Acta Pharm. Technol. 34(3), pp. 129-139 (1988), the contents of which are incorporated herein by reference.

Kits according to the invention can include a container containing the sterile lyophilised material and enclosing the gas or gas mixtures described above, in one chamber under reduced pressure. In a preferred embodiment, the lyophilised material and the gas are contained in a first chamber of said container. The sterile aqueous liquid may be contained in a different container or preferably in a second separate chamber of the same container. In one embodiment, the container is a conventional septum-sealed vial. In another, it has a means for directing or permitting application of sufficient bubble forming energy into the contents of the container. Where the dried product is contained in a vial this is conveniently sealed with a septum through which the carrier liquid may be injected using an optionally prefilled syringe. Alternatively, the dried product and carrier liquid may be supplied together in a two-component kit such as a dual chamber syringe. It may be advantageous to mix or gently shake the product following reconstitution. However, as noted above, in the stabilised contrast agents according to the invention the size of the gas microbubbles are substantially independent from the amount of agitational energy applied to the reconstituted dried product. Accordingly no more than gentle hand shaking may be required to give reproducible products with consistent microbubble size. It can be appreciated by one ordinary skilled in the art that other combinations of separate containers or of two-chamber reconstitution systems capable of combining the dried powder and the gas with the aqueous solution in a sterile manner are also within the scope of the present invention. In such systems, it is particularly advantageous if the aqueous phase can be interposed between the water-insoluble gas and the environment, to increase shelf life of the product. Where a material necessary for forming the microbubble is not already present in the container, it can be packaged with the other components of the kit, preferably in a form or container adapted to facilitate ready combination with the other components of the kit.

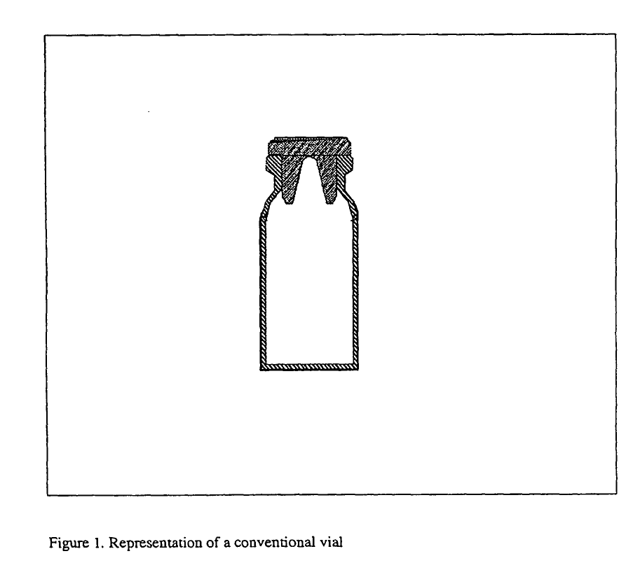

Suitable containers, vials or bottles according to the present invention are for example illustrated in FIG. 1. No specific containers, vial or connection systems are required; the present invention may use conventional containers, vials and adapters. The only requirement is a good gas-seal between the stopper and the container. The quality of the seal, therefore, becomes a matter of primary concern; any degradation of seal integrity could allow undesirables substances to enter the vial. In addition to assuring sterility, vacuum retention is essential for products stoppered at reduced pressures to assure safe and proper reconstitution. The material of the stopper forming the gas-seal of the container can be an elastomeric compound or multicomponent formulation based on an elastomer, such as poly(isobutylene) or butyl rubber. Conveniently a butyl rubber stopper from Daiko Seiko ltd. can be used.

The dry formulations of the invention can be prepared by very simple methods. Conveniently, the dried material of the contrast agent of the invention can, for example, be prepared with non-laminarized film-forming surfactants such as phospholipids or with film-forming surfactants, which were lamellarized, or laminarized prior to their contacting with air or another gas. The terms lamellar or lamella or laminar form indicates that the surfactants are in the form of thin films or sheets involving one or more monomolecular layer(s). As described in WO 91/15244 conversion of film-forming surfactants into lamellar form can easily be done for example by any liposome forming method for instance by high-speed mechanical homogenisation or by sonication under acoustical or ultrasonic frequencies. One method is to first prepare an aqueous solution in which the film forming lipids are laminarized, for instance by sonication, or using any conventional technique commonly used in the liposome field, this solution also containing the other desired additives, i.e. viscosity enhancers, non-film forming surfactants, electrolyte, etc., and thereafter freeze drying to a free flowable powder which is then stored in the presence of air or an entrappable gas.

According to an alternative embodiment (described for instance in the above cited U.S. Pat. No. 5,597,549) a film forming surfactant and a hydrophilic stabiliser (e.g. polyethylene glycol, polyvinyl pyrrolidone, polyvinyl alcohol, glycolic acid, malic acid or maltol) can be dissolved in an organic solvent (e.g. tertiary butanol, 2-methyl-2-butanol or C₂C₁₄F₂) and the solution can be freeze-dried to form a dry powder. Alternatively, as disclosed in the above cited co-pending EP application EP 03002375.8, a phospholipid and a lyoprotecting agent (e.g. carbohydrates, sugar alcohols, polyglycols and mixtures thereof) can be dispersed in an emulsion of water with a water immiscible organic solvent (e.g. branched or linear alkanes, alkenes, cyclo-alkanes, aromatic hydrocarbons, alkyl ethers, ketones, halogenated hydrocarbons, perfluorinated hydrocarbons or mixtures thereof) and the obtained emulsion can be lyophilzed to obtain a lyophilized material.

Freeze-drying and spray drying techniques can be used to obtain the dried contrast agent of the present invention. The dried or lyophilised product will generally be in powder or in a cake form and is readily reconstitutable in a suitable aqueous liquid carrier, which is physiologically acceptable, sterile and injectable. Suitable liquid carriers are water, aqueous solutions such as saline (which may advantageously be balanced so that the final product for injection is not hypotonic), or solutions of one or more tonicity adjusting substances such as salts or sugars, sugar alcohols, glycols or other non-ionic polyol materials (eg. glucose, sucrose, sorbitol, mannitol, glycerol, polyethylene glycols, propylene glycols and the like). In practice, all injectable compositions after reconstitution of the lyophilised contrast agent should also be as far as possible isotonic with blood. Hence, before injection, small amounts of isotonic agents may also be added to the suspensions of the invention. The isotonic agents are physiological solutions commonly used in medicine and they comprise aqueous saline solution (0.9% NaCl), 2.6% glycerol solution, 5% dextrose solution, etc. Reconstitution will generally require only minimal agitation such as may, for example, be provided by gentle hand shaking. The size of the microbubbles so generated is consistently reproducible and in practice is independent of the amount of agitational energy applied. Thus, the reconstitution of aqueous microbubbles suspensions is obtained by simple dissolution of the gas-stored dried film forming surfactant without any violent agitation.

The volume and concentrations of the reconstitution liquid may desirably be balanced to make the resulting ready-to-use formulations substantially isotonic. Hence the volume and concentration of reconstitution fluid chosen will be dependent on the type and amount of stabilizer (and other bulking agents) present in the freeze-dried product.

For ultrasound applications such as echocardiography, in order to permit free passage through the pulmonary system and to achieve resonance with the preferred imaging frequencies of about 0.1-15 MHz, microbubbles having an average size of 0.1-10 μm are necessary.

Contrast agents according to the present invention may be produced with a very narrow size distribution within the range preferred for echocardiography, thereby greatly enhancing their effective echogenicity as well as their safety in vivo, and rendering the contrast agents of particular advantage in applications such as blood pressure measurements, blood flow tracing and ultrasound tomography.

The contrast agents obtainable by the process of the present invention may be used in other diagnostic imaging techniques, including in particular Magnetic Resonance. Possible other diagnostic imaging applications include scintigraphy, light imaging, and X-ray (including X-ray phase imaging.

The bubble suspensions of the present invention are also useful in other medical/diagnostic applications where it is desirable to target the stabilized microbubbles to specific sites in the body following their injection, for instance to thrombi present in blood vessels, to atherosclerotic lesions (plaques) in arteries, to tumor cells, as well as for the diagnosis of altered surfaces of body cavities, e.g. ulceration sites in the stomach or tumors of the bladder. For this, one can bind monoclonal antibodies tailored by genetic engineering, antibody fragments, peptides, oligopeptides or polypeptides designed to mimic antibodies, bioadhesive polymers, lectins and other site-recognizing molecules (ligands) to the phospholipid film-forming surfactant stabilizing the microbubbles. Thus phospholipid derivatives may be obtained i.e. DPPE coupled with N-biotinyl in example 2.

The foregoing description will be more fully understood with reference to the following Examples. Such Examples, are, however, exemplary of methods of practising the present invention and are not intended to limit the scope of the invention.

### EXAMPLES

For lipid components, following abbreviations are used in the description of the Examples:
DAPC diarachidoyl phosphatidyl choline
DCP dicetyl phosphate
DPPC dipalmitoyl phosphatidyl choline
DPPG dipalmitoyl phosphatidyl glycerol
DPPA dipalmitoyl phosphatidic acid
DPPS dipalmitoyl phosphatidyl serine
DSPC distearoyl phosphatidyl choline
DSPS distearoyl phosphatidyl serine
DSPE-PEG2000 distearoyl phosphoethanolamine-N-poly(ethylene glycol)2000
DPPE-PEG5000 dipalmitoyl phosphoethanolamine-N-poly(ethylene glycol)5000
N-Biotinyl Cap-PE n-biotinyl caproyl phosphatidylethanolamine
DSTAP distearoyl trimethylammonium propane

All of these phospholipids were purchased from Lipoid (Switzerland) and/or from Avanti Polar Lipids (USA).

### Example 1

A solution containing 0.9 g of DPPC and 100 mg of DPPG was prepared with 50 ml of hexane/isopropanol 8/2 v/v; (Fluka, Switzerland). The solvents were evaporated to dryness. 20 ml of distilled water were added and the mixture was heated at 65° C. for one hour on a rotavapor apparatus. The resulting suspension was then extruded through successively 3 and 1 μm polycarbonate membranes (Nuclepore®). After cooling, the extruded suspension was mixed with Macrogol 4000 solution (100 mg/ml; Macrogol 4000 is poly (ethylene glycol) with a molecular weight of 4000 and purchased from Clarian, Germany) with a volume ratio of 1:9 (Lipid suspension/Macrogol solution) and rapidly frozen at −45° C. in a round glass flask. The frozen sample was then freeze-dried under vacuum (0.2 mbar) and overnight. Aliquots of the obtained lyophilisate were placed in 10 ml glass vials (450 mg/vial) and sealed with a gas tight stopper. The vials were totally evacuated by high vacuum pump and then filled with C₄F₁₀ gas under different absolute gas pressures (100, 200, 300, 500 and 1000 mbar). The lyophilisate samples were then reconstituted with 5 ml saline solution (injected through the stopper) by vigorous shaking on a vortex mixer to generate gas microbubbles. Coulter counter measurements (Multisizer II) were performed on the bubble suspensions to evaluate the bubble characteristics (size and number) under different reduced gas pressures. The results of Coulter measurements are summarized below. Dn corresponds to the mean diameter in number. The bubble volume is the total volume of gas entrapped in the bubbles per ml of reconstituted solution.

TABLE 1

| Pressure | Total bubble | | Bubble volume |
|---|---|---|---|
| (mbar) | conc. (10⁹/ml) | Dn (μm) | (μl/ml) |
| 1000 | 1.50 | 1.8 | 20.0 |
| 900 | 1.30 | 2.0 | 20.5 |
| 500 | 0.87 | 2.0 | 22.1 |
| 300 | 0.41 | 2.2 | 13.6 |
| 200 | 0.42 | 2.2 | 11.6 |
| 100 | 0.23 | 2.4 | 6.0 |

These results surprisingly show that with a reduced pressure of 500 mbar, gas microbubbles properties are still fairly good as compared to the sample prepared under normal atmosphere pressure (1000 mbar). Even at a pressure as low as 100 mbar, more than 2×10⁸ bubbles/ml are obtained.

### Example 2

A series of aqueous phospholipid suspensions were prepared with the following lipid compositions:
A. 25 mg of DAPC, 70 mg of DSPS and 5 mg of DSPE-PEG2000
B. 45 mg of DSPC, 45 mg of DPPG, 10 mg of palmitic acid
C. 54 mg DPPC, 6 mg DPPA, 40 mg PE-PEG5000
D. 100 mg of DPPS
E. 90 mg of DPPS, 10 mg of DSPE-PEG2000
F. 50 mg DPPC, 45 mg DPPS, 5 mg Pluronic® F108
G. 50 mg of DPPG, 150 mg of Pluronic® F₆₈, 2.5 mg of N-biotinyl Cap-PE
H. 90 mg DSPC, 10 mg TAP

The components of each composition were dispersed in 50 ml aqueous solution containing 1.5 g of glycerol and 5 g of propylene glycol. All dispersions were heated 1 hour at 75° C. on a rotavapor apparatus for lipid hydration. After cooling, the suspensions were then homogenized under high-speed mechanical agitation using a Polytron® (Kinematica AG, 15'000 rpm and 2 min.) at 5° C. and under C4F10 gas in air. The generated bubble suspensions were introduced in 50 ml syringes and decanted for one night. The syringes were kept horizontally. After decantation (a white opaque thin bubble layer forms on the top of the solution), the sub-phase (˜45 ml) was discarded, the bubble-containing phase (˜5 ml) was recovered and resuspended in the same volume of a 5% Dextran-15 (Mw 15000) aqueous solution. This bubble decantation process allows to eliminate the excess lipids not associated with bubbles and thus produce bubble suspensions containing very low phospholipid concentrations (see U.S. Pat. No. 5,445,813 incorporated herein by reference in its entirety). The resulting bubble suspensions were, introduced in 10 ml glass vials (1 ml per vial) then rapidly frozen at −45° C. (see U.S. Pat. No. 5,961,956 incorporated herein by reference) and freeze-dried. After lyophilisation, the vials were evacuated by high vacuum pump and then filled with C4F10 gas, either under normal (1000 mbar) or reduced gas pressure (500 mbar) and sealed with gas tight stoppers. Microbubble suspensions were obtained by injecting through the stopper 0.9% NaCl (5 ml) to each vial followed by vigorous agitation. Coulter counter measurements were performed on all bubble suspensions. The results are summarized below:

TABLE 2

| | | |
|---|---|---|
| Total | Number mean | Total |
| bubble conc. | diameter Dn | bubble volume |
| (10⁸/ml) | (μm) | (μl/ml) |

| | 1000 | 500 | 1000 | 500 | 1000 | 500 |
|---|---|---|---|---|---|---|
| Sample | mbar | mbar | mbar | mbar | mbar | mbar |
| A | 7.1 | 7.0 | 2.2 | 2.1 | 8.6 | 8.1 |
| B | 2.7 | 2.5 | 2.0 | 2.0 | 7.7 | 8.0 |
| C | 2.1 | 1.5 | 2.4 | 2.3 | 4.5 | 2.8 |
| D | 6.6 | 8.6 | 2.0 | 2.1 | 5.6 | 6.9 |
| E | 4.3 | 4.3 | 2.4 | 2.3 | 10.0 | 9.7 |
| F | 2.7 | 2.5 | 2.1 | 2.2 | 7.5 | 6.9 |
| G | 2.0 | 1.8 | 2.0 | 2.0 | 5.5 | 5.3 |
| H | 0.3 | 0.3 | 2.3 | 2.3 | 3.4 | 3.1 |

These results show that even under half the normal gas pressure (500 mbar vs. 1000 mbar), the tested formulations maintain the properties of the microbubbles (bubble concentrations, mean sizes and gas encapsulated volumes).

### Example 3

A phospholipid mixture containing 27 mg of DPPC, 3 mg of DPPA and 20 mg of DPPE-PEG5000 was dissolved in 18 g of tert-butanol under reflux (82° C.). After dissolution, 3 g of Macrogol 4000 were added. After complete dissolution, aliquots of the solution were filled into 10 ml glass vials (310 μl/vial), frozen at −45° C. and lyophilized. The lyophilisate-containing vials were evacuated by high vacuum pump, filled with various gases (see below) under different absolute gas pressures (100, 300, 500, 700 and 1000 mbar) and sealed with gas tight stoppers. The lyophilisate samples were reconstituted with 5 ml saline solution (injected through the stopper) by vigorous shaking to generate gas microbubbles. Coulter counter analyses were performed and the results are presented in Table 3. The concentration of gas microbubbles prepared at 300, 500 and 700 mbar are expressed as relative bubble concentrations, normalized with the values obtained from samples prepared at 1000 mbar (atmospheric pressure).

TABLE 3

| | Bubble conc. (%) | | | | |
|---|---|---|---|---|---|
| | 1000 | 700 | 500 | 300 | 100 |
| | mbar | mbar | mbar | mbar | mbar |
| Air | 100% (1.3 × 10⁸/ml) | 68% | 23% | 11% | 0% |
| SF₆ | 100% (3.7 × 10⁸/ml) | 83% | 45% | 17% | 3% |
| C₂F₆ | 100% (3.2 × 10⁸/ml) | 96% | 80% | 56% | 16% |
| C₄F₁₀ | 100% (3.8 × 10⁸/ml) | 93% | 79% | 70% | 27% |
| C₂F₆/C₄F₁₀ | 100% (4.0 × 10⁸/ml) | 76% | 66% | 52% | 27% |
| (90:10) | | | | | |
| C₄F₁₀/air | 100% (3.2 × 10⁸/ml) | 85% | 73% | 59% | 25% |
| (35:65) | | | | | |
| C₃F₈/air | 100% (3.6 × 10⁸/ml) | 104% | 79% | 63% | 31% |
| (65:35) | | | | | |

The nature of the gas or gas mixture appears to have an important effect on the microbubble concentration under reduced gas pressure, especially at the low-pressure values (100-500 mbar). It should be noted that a product containing 10⁷ bubbles/ml can be useful as echographic contrast agent.

### Example 4

Lyophilisates were prepared and reconstituted as described in Example 3 except for the phospholipid composition: 47.5 mg of DSPC, 2.5 mg of DCP were used here (instead of DPPC/DPPA/DPPE-PEG5000. The Coulter counter results are given in Table 4.

TABLE 4

| | Bubble conc. (%) | | | | |
|---|---|---|---|---|---|
| | 1000 | 700 | 500 | 300 | 100 |
| | mbar | mbar | mbar | mbar | mbar |
| Air | 100% (1.2 × 10⁸/ml) | 31% | 21% | 9% | 0% |
| SF₆ | 100% (3.0 × 10⁸/ml) | 76% | 50% | 26% | 2% |
| C₂F₆ | 100% (3.5 × 10⁸/ml) | 58% | 56% | 43% | 8% |
| C₄F₁₀ | 100% (3.9 × 10⁸/ml) | 74% | 54% | 53% | 12% |
| C₂F₆/C₄F₁₀ | 100% (3.5 × 10⁸/ml) | 73% | 64% | 47% | 19% |
| (90:10) | | | | | |
| C₄F₁₀/air | 100% (3.2 × 10⁸/ml) | 97% | 79% | 64% | 17% |
| (35:65) | | | | | |
| C₃F₈/air | 100% (4.0 × 10⁸/ml) | 88% | 73% | 49% | 9% |
| (65:35) | | | | | |

### Example 5

Lyophilisates were prepared as described in Example 3 using 25 mg of DPPC, 25 mg of DPPG (instead of DPPC, DPPA and DPPE-PEG5000). Aliquots of the tert-butanolic solution (500 μl/vial) were frozen and lyophilized. The lyophilisate-containing vials were evacuated and filled with various gases (see below) under different reduced gas pressures (100, 300, 500, 700, 1000 mbar) and sealed with gas tight stoppers. The lyophilized samples were reconstituted with 5 ml saline and Coulter analysis was performed. The results are presented in Table 5.

TABLE 5

| | Bubble conc. (%) | | | | |
|---|---|---|---|---|---|
| | 1000 | 700 | 500 | 300 | 100 |
| | mbar | mbar | mbar | mbar | mbar |
| Air | 100% (0.46 × 10⁸/ml) | 25% | 20% | 9% | 0% |
| SF₆ | 100% (1.8 × 10⁸/ml) | 54% | 48% | 9% | |
| C₂F₆ | 100% (4.7 × 10⁸/ml) | 40% | 39% | 27% | 8% |
| C₄F₁₀ | 100% (3.1 × 10⁸/ml) | 89% | 58% | 50% | 8% |
| C₂F₆/C₄F₁₀ | 100% (3.3 × 10⁸/ml) | 72% | 59% | 42% | 13% |
| (90:10) | | | | | |
| C₄F₁₀/air | 100% (2.8 × 10⁸/ml) | 52% | 56% | 54% | 11% |
| (35:65) | | | | | |
| C₃F₈/air | 100% (2.8 × 10⁸/ml) | 80% | 66% | 49% | 23% |
| (65:35) | | | | | |

### Example 6

Lyophilisates were prepared and reconstituted as described in Example 3 using 47.5 mg of DAPC, 2.5 mg of DPPG (instead of DPPC, DPPA and DPPE-PEG5000). Aliquots of the tert-butanolic solution (250 μl/vial) were frozen and lyophilized (instead of 310 μl/vial in Example 3). The results of Coulter analysis are presented in Table 6 for each gas or gas mixture-containing formulation tested.

TABLE 6

| | Bubble conc. (%) | | | | |
|---|---|---|---|---|---|
| | 1000 | 700 | 500 | 300 | 100 |
| | mbar | mbar | mbar | mbar | mbar |
| Air | 100% (0.8 × 10⁸/ml) | 62% | 23% | 0% | |
| SF₆ | 100% (4.7 × 10⁸/ml) | 70% | 70% | 40% | 1% |
| C₂F₆ | 100% (3.1 × 10⁸/ml) | 118% | 118% | 106% | 11% |
| C₄F₁₀ | 100% (3.2 × 10⁸/ml) | 128% | 148% | 90% | 23% |
| C₂F₆/C₄F₁₀ | 100% (5.4 × 10⁸/ml) | 60% | 63% | 54% | 18% |
| (90:10) | | | | | |
| C₄F₁₀/air | 100% (3.6 × 10⁸/ml) | 108% | 115% | 74% | 15% |
| (35:65) | | | | | |
| C₃F₈/air | 100% (5.6 × 10⁸/ml) | 91% | 80% | 49% | 12% |
| (65:35) | | | | | |

### Example 7

Lyophilisates were prepared and reconstituted as described in Example 6 with 22.5 mg of DSPC, 22.5 mg of DPPG and 5 mg of palmitic acid (instead of DAPC, DPPG). The Coulter counter results are given in Table 7.

TABLE 7

| | Bubble conc. (%) | | | | |
|---|---|---|---|---|---|
| | 1000 | 700 | 500 | 300 | 100 |
| | mbar | mbar | mbar | mbar | mbar |
| Air | 100% (1.2 × 10⁸/ml) | 49% | 22% | 11% | 0% |
| SF₆ | 100% (2.3 × 10⁸/ml) | 106% | 48% | 22% | 16% |
| C₂F₆ | 100% (2.7 × 10⁸/ml) | 96% | 79% | 50% | 22% |
| C₄F₁₀ | 100% (3.1 × 10⁸/ml) | 99% | 96% | 58% | 29% |
| C₂F₆/C₄F₁₀ | 100% (2.9 × 10⁸/ml) | 94% | 83% | 67% | 33% |
| (90:10) | | | | | |
| C₄F₁₀/air | 100% (2.7 × 10⁸/ml) | 109% | 85% | 64% | 29% |
| (35:65) | | | | | |
| C₃F₈/air | 100% (3.0 × 10⁸/ml) | 83% | 86% | 57% | 52% |
| (65:35) | | | | | |

### Example 8

Lyophilisates were prepared and reconstituted as described in Example 6 with 25 mg of DSPC, 22.5 mg of DPPG and 2.5 mg of Pluronic® F108 (instead of DAPC, DPPG). The Coulter counter results are given in Table 8.

TABLE 8

| | Bubble conc. (%) | | | | |
|---|---|---|---|---|---|
| | 1000 | 700 | 500 | 300 | 100 |
| | mbar | mbar | mbar | mbar | mbar |
| Air | 100% (3.0 × 10⁸/ml) | 56% | 38% | 17% | 0% |
| SF₆ | 100% (2.6 × 10⁸/ml) | 111% | 54% | 61% | 5% |
| C₂F₆ | 100% (4.4 × 10⁸/ml) | 92% | 66% | 69% | 15% |
| C₄F₁₀ | 100% (4.3 × 10⁸/ml) | 105% | 101% | 76% | 21% |
| C₂F₆/C₄F₁₀ | 100% (4.7 × 10⁸/ml) | 89% | 90% | 67% | 28% |
| (90:10) | | | | | |
| C₄F₁₀/air | 100% (4.6 × 10⁸/ml) | 89% | 89% | 73% | 14% |
| (35:65) | | | | | |
| C₃F₈/air | 100% (5.1 × 10⁸/ml) | 85% | 86% | 70% | 24% |
| (65:35) | | | | | |

Examples 3 to 8 show that the bubble formation under reduced gas pressures (<1000 mbar) can be further improved or controlled by selecting the composition of the lipids and the gas or gas mixtures (solubility, molecular weight).

The following examples (Example 9-11) show that the use of a reduced gas pressure may help to control the microbubble size distribution, especially to eliminate large microbubbles (>8 μm). This is important to reduce the risk of gas embolism in blood vessels and to improve transpulmonary passage of microbubbles thus allowing reproducible and quantitative in vivo ultrasonic imaging properties.

### Example 9

Lyophilisates were prepared as described in Example 8 and filled with the gas C4F10 at various reduced gas pressures (see Table 9). [How were these reconstituted?] The Coulter counter analyses were performed to determine bubble characteristics such as bubble concentration (Nb/ml), mean diameter in number (Dn) and in volume (Dv) and the bubble volume present in bubbles larger than 8 μm (vol. >8 μm) expressed as a percentage of the bubble volume in bubbles larger than 8 μm present at a pressure of 1000 mbar. The results are given in Table 9.

TABLE 9

| | Pressure (mbar) | | | | | |
|---|---|---|---|---|---|---|
| | 1000 | 700 | 500 | 300 | 200 | 100 |
| Nb/ml (×10⁸) | 4.7 | 4.2 | 4.2 | 3.1 | 2.6 | 2.6 |
| Dn (μm) | 2.3 | 2.3 | 2.2 | 2.2 | 2.1 | 2.0 |
| Dv (μm) | 13.4 | 10.7 | 8.8 | 7.4 | 7.3 | 5.6 |
| Dv/Dn* | 5.8 | 4.7 | 3.9 | 3.4 | 3.5 | 2.8 |
| Vol. > 8 μm (%) | 100 | 58 | 35 | 18 | 10 | 4 |

*Dv/Dn = Polydispersity index

This example clearly shows that without significantly changing the general bubble characteristics (Nb/ml, Dn), the use of a reduced pressure allows to control of the bubble size upon reconstitution of the powder and to considerably reduce the number of large microbubbles (>8 μm). Thus the number of bubbles produced under a pressure of 100 mbar is 55% of the value obtained at 1000 mbar (2.6×10⁸ vs 4.7×10⁸) but they contain 25 times less volume (Table 9 shows that the total volume in microbubbles larger than 8 μm is reduced from 100% at 1000 mbar to 4% at 100 mbar).

### Example 10

Lyophilisates were prepared and reconstituted as described in Example 8 and filled with a gas mixture of C3F8 and air (65:35% by volume) at various reduced gas pressures. Coulter analyses were performed to determine the bubble characteristics such as the bubble concentration (Nb/ml), the mean diameter in number (Dn), the mean diameter in volume (Dv) and the bubble volume present in bubbles larger than 8 μm as defined in example 9. The results are given in Table 10.

TABLE 10

| | Pressure (mbar) | | | | | |
|---|---|---|---|---|---|---|
| | 1000 | 700 | 500 | 300 | 200 | 100 |
| Nb/ml (×10⁸) | 5.1 | 4.3 | 4.4 | 3.6 | 3.1 | 1.2 |
| Dn (μm) | 2.3 | 2.2 | 2.2 | 2.1 | 2.0 | 1.9 |
| Dv (μm) | 12.9 | 13.8 | 10.4 | 7.4 | 7.1 | 5.0 |
| Dv/Dn* | 5.7 | 6.2 | 4.8 | 3.5 | 3.6 | 2.7 |
| Vol. > 8 μm (%) | 100 | 90 | 47 | 17 | 10 | 3 |

More than 50% of the of the volume present in large microbubbles (>8 μm) is eliminated by using a reduced pressure of 500 mbar when compared with the sample prepared at the atmospheric gas pressure (1000 mbar). The other bubble properties remain substantially unchanged. The reduction is even more impressive at 200 mbar where the volume in large (>8 μm) bubbles falls to 10% of the value at atmospheric pressure whereas the total bubble concentration drops only by 39% (from 5.1 to 3.1×10⁸/ml).

### Example 11

A phospholipid containing spray-dried formulation was obtained as follows:

0.45 g of hydrogenated egg phosphophatidylcholine (Lipoid EPC-3) and 0.15 g of Poloxamer 188 were dispersed in 100 ml of distilled water. The dispersion was heated 1 hour at 50° C. then cooled down to 4° C. 3 ml of 1,1,2 trichlorotrifluoroethane (Freon® 113) was mixed with the dispersion using high speed homogenization (Polytron®, 2 min at 10'000 rpm and 4° C.). The resulting emulsion was placed in a refrigerator 1 night then extruded through a Microfluidizer (Microfluidics Corp.) to reduce emulsion size (10'000 psi, 5° C. and 5 pass). An aqueous solution containing 3.6 g of maltodextrin, 3 g of NaCl and 2.6 g of Na₂HPO₄ and 0.9 g of NaH₂PO₄ was prepared in 100 ml distilled water. This solution was mixed with the emulsion and then spray-dried (Mini Spray dryer Büchi 190: inlet air temperature, 235° C.; outlet temperature, 105° C.).

The resulting dried powders were aliquoted into 10 ml glass vials, filled with C4F10 under various gas pressures and then reconstituted with distilled water (30 mg/ml). Coulter counter analyses were performed as before. The bubble characteristics obtained (Nb/ml, Dn, Dv, polydispersity index and the percentage of the bubble volume (>8 μm)) are summarized in Table 11.

TABLE 11

| | Pressure (mbar) | | | | |
|---|---|---|---|---|---|
| | 1000 | 700 | 500 | 300 | 200 |
| Nb/ml (×10⁸) | 9.1 | 9.5 | 7.8 | 8.3 | 6.2 |
| Dn (μm) | 2.6 | 2.5 | 2.6 | 2.4 | 2.4 |
| Dv (μm) | 14.5 | 13.1 | 11.4 | 8.6 | 9.6 |
| Dv/Dn* | 5.5 | 5.3 | 4.4 | 3.6 | 4.0 |
| Vol. > 8 μm (%) | 100 | 71 | 46 | 26 | 17 |

*Polydispersity index

Here again, the bubble size distribution is considerably narrowed by the use of a reduced gas pressure. This is especially true for the reduction in large microbubbles (>8 μm). At 200 mbar for instance, there are almost as many bubbles as at atmospheric pressure (8.3 vs 9.1×10⁸/ml) but the volume of large bubbles (>8 μm) is reduced by 74%. Thus the present invention allows improvement of the safety profile of this formulation.

### Example 12

(In Vivo Imaging)

This example shows the in vivo efficacy of the formulation of Example 11 (spray-dried phospholipid suspension) prepared under various reduced pressures. Briefly, overnight fasted minipigs were anaesthetized with Stressnil (Azaperone, Janssen: 2 mg/kg) followed by Forene (Isofurane, Abbot). Left heart ventricle opacification was evaluated using an ATL HDI-5500 imaging system equipped with Pulse Inversion mode. The P4-2 probe (2.8/3.2 MHz) was used. Imaging dose (0.1 ml/kg) and all setting parameters were optimized at the beginning of the experiment (acoustic power, gain, depth, focus, frame rates and processing) and kept constant for all tested samples. The results (peak intensity, duration and area under the intensity vs. time curve) are given in Table 12.

TABLE 12

| | max. | Contrast | |
|---|---|---|---|
| Tested | Intensity | duration | Area under |
| sample | (PI) | (min) | the curve |
| 1000 mbar | 85 | 5.3 | 6800 |
| (ref.) | | | |
| 900 mbar | 84 | 5.4 | 6810 |
| 500 mbar | 80 | 5.4 | 6785 |
| 300 mbar | 72 | 4.9 | 6110 |
| 200 mbar | 81 | 4.6 | 5760 |
| 100 mbar | 72 | 4.1 | 4300 |

The in vivo results show that even at 100 mbar of the reduced gas pressure, the formulation is still highly effective. Similar results were obtained for contrast enhancement studies of the myocardium.

### Example 13

(Contrast Agent Preparation from a Kit)

The stability of gas microbubbles prepared under various reduced gas pressures was evaluated. Phospholipid containing lyophilisates as described in Example 7 were prepared in glass vials (DIN8R) and filled with 55% of C4F10 in air at 500 and 1000 mbar. A pre-filled saline syringe (containing 5 ml of NaCl-0.9% solution) was connected to the vial for the reconstitution. For the 1000 mbar sample (at 1 atmosphere), a Bio-Set® system equipped with a vent filter was used to compensate the overpressure resulting from the addition of the volume of saline into the vial; while for 500 mbar sample, no vent was necessary. As the gas pressure in the vial is only half of the atmospheric pressure, when the syringe is connected to the vial, the vacuum (500 mbar) automatically sucks in the saline solution until the liquid (5 ml) is totally aspired. At this point, the gas pressure in the vial was restored to atmospheric pressure (1000 mbar) by simple reconstitution. To compare the stability of microbubbles suspensions initially prepared under the normal (1000 mbar) and reduced (500 mbar) pressures, Coulter analyses were performed at t=0 and 6 h after the reconstitution. The results (comparison of the bubble concentration and volume between t=0 and 6 h after reconstitution) are summarized in Table 13.

TABLE 13

| | |
|---|---|
| Bubble conc. | Bubble vol. |
| (Nb/ml) | (μl/ml) |

| | (t = 0) | (t = 6 h) | (t = 0) | (t = 6 h) |
|---|---|---|---|---|
| 1000 mbar* | 2.6 × 10⁸ | 2.6 × 10⁸ | 17.7 | 13.7 |
| 500 mbar* | 2.0 × 10⁸ | 1.9 × 10⁸ | 10.5 | 10.1 |

*Tested samples (n = 5)

The results show that the stability of the gas microbubbles suspensions prepared at 500 mbar and 1000 mbar are comparable.

### Example 14

(Stability of the Lyophilisate Under Reduced Gas Pressure During Storage)

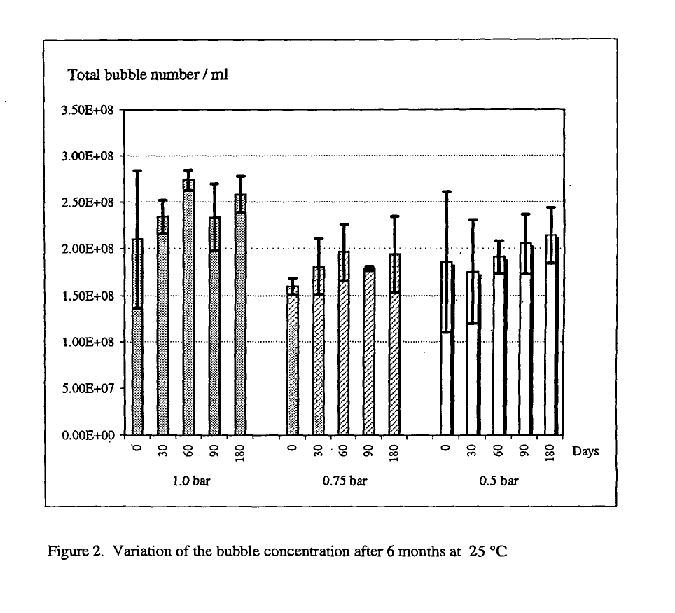

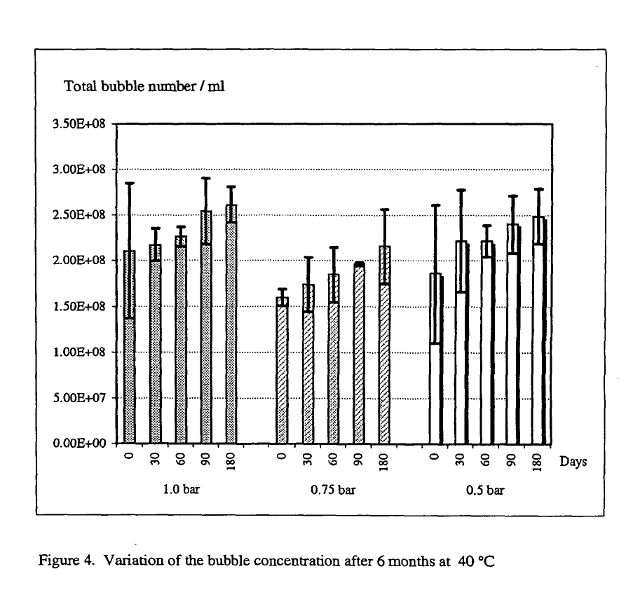

Phospholipid containing lyophilisates described in Example 7 were prepared in glass vials (DIN8R) and filled with 55% of C4F10 in air with gas pressures respectively at 500, 750 and 1000 mbar. The stability of lyophilized samples under the reduced gas pressures was evaluated after 0, 1, 3 and 6 months of storage at 25° C. and 40° C. The Coulter analyses (bubble concentration) are given below (Table 14 and FIGS. 2 and 4).

TABLE 14

| | | t = 1 month | | t = 3 months | |
|---|---|---|---|---|---|
| | t = 0 | 25° C. | 40° C. | 25° C. | 40° C. |
| 1000 mbar* | 2.1 × 10⁸ | 2.3 × 10⁸ | 2.2 × 10⁸ | 2.3 × 10⁸ | 2.5 × 10⁸ |
| 750 mbar* | 1.6 × 10⁸ | 1.8 × 10⁸ | 1.7 × 10⁸ | 1.8 × 10⁸ | 2.0 × 10⁸ |
| 500 mbar* | 1.9 × 10⁸ | 1.8 × 10⁸ | 2.2 × 10⁸ | 2.0 × 10⁸ | 2.4 × 10⁸ |

*Tested samples (n = 5)

The Coulter counter results showed no significant differences between the three lyophilized samples filled with different gas pressures after 1 and 3 months of storage.

The resistance to pressure was evaluated in vitro using a spectrophotometric method. The bubble suspension was diluted 1/50 in a solution of 0.9% sodium chloride and introduced in the sample cell of a spectrophotometer (Jenway 6100 Model). The cell was connected to a pneumatic valve allowing a linear pressure increase from 0 to 700 mmHg. The simultaneous recording of the pressure (via a pressure sensor) and of the absorbance provides typical curves allowing the determination of a “critical pressure” (Pc50) corresponding to the pressure at which the absorbance has decreased by 50%.

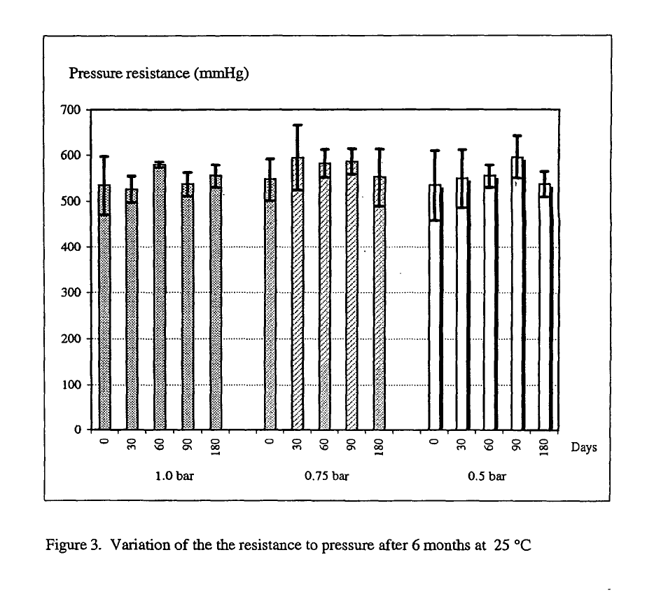

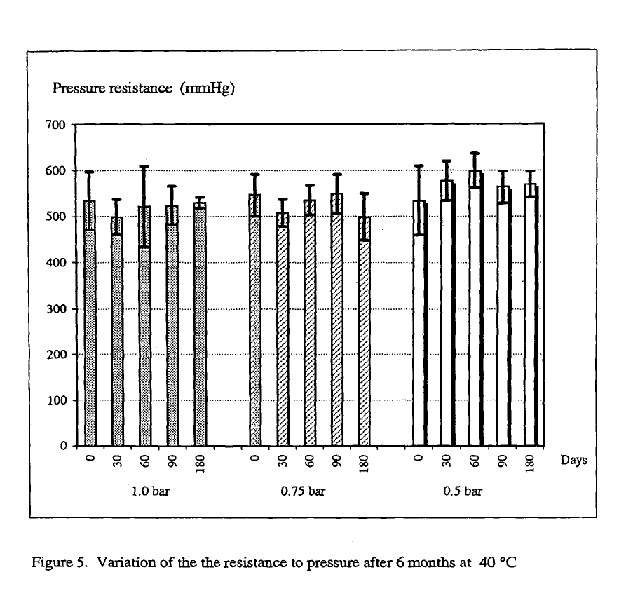

The resistances to pressure shown in FIGS. 3 and 5 are highly constant whatever the storage conditions. Temperature and pressure inside the vial have no influence on this parameter.

In conclusion, there is not a significant influence of the duration and the temperature of storage on the bubble characteristics (pressure resistance, bubble concentration).

### Example 15

Vials containing composition C were obtained as described in Example 2. After freeze drying, the vials were exposed to polarized helium gas (obtained from the Laue-Langevin Institute, Grenoble France) both under normal (1000 mbar) and reduced gas pressures (500 and 100 mbar). Bubble suspensions were obtained after injection of saline (1 ml) through the stoppers as described in example 2. The bubble suspensions (1 ml) were injected in the jugular vein of rats and imaged in a 2 Tesla MR imager with a 17 cm bore magnet and 6 cm diameter volumic coil tunable to both ¹H and ³He resonance frequency. The abdominal aorta was clearly visible. No significant differences between the vials with different gas pressures were detected.

1. A contrast agent for diagnostic imaging in the form of an aqueous suspension of microvesicles of a gas stabilized by a layer of film-forming surfactant, said suspension being obtainable by reconstitution in a physiologically acceptable aqueous carrier of a dried material comprising at least one film-forming surfactant and being exposed to a gas, wherein, prior to said reconstitution, said material is exposed to an atmosphere of said gas having a pressure lower than atmospheric pressure.

2. A contrast agent according to claim 1 wherein the pressure of said gas is from about 100 to about 900 mbar.

3. A contrast agent according to claim 1 wherein the pressure of said gas is from about 300 to 700 mbar.

4. A contrast agent according to claim 1 wherein the pressure of said gas is from about 400 to 600 mbar.

5. A contrast agent according to claim 1 wherein the dried material is a lyophilized material.

6. A contrast agent according to claim 1 wherein said dried material is contained in a container which undergoes to vacuum before the gas is contacted with said material.

7. A contrast agent according to claim 1 wherein the gas is selected from the group consisting of halogenated gases, air, nitrogen, carbon dioxide, helium, krypton, xenon, argon, methane, hyperpolarized gases, hyperpolarized noble gases, radioactive gases and mixtures thereof.

8. A contrast agent according to claim 7 wherein the halogenated gas is a perfluorinated gas.

9. A contrast agent according to claim 8 wherein the perfluorinated gas is selected from the group consisting of perfluoromethane, perfluoroethane, perfluoropropane, perfluorobutanes, perfluoropentanes, perfluorohexanes, perfluoroheptanes, perfluoropropene, perfluorobutenes, perfluorobutadiene, perfluorobut-2-yne, perfluorocyclobutane, perfluoromethylcyclobutane, perfluorodimethylcyclobutanes, perfluorotrimethylcyclobutanes, perfluorocyclopentane, perfluoromethylcyclopentane, perfluorodimethylcyclopentanes, perfluorocyclohexane, perfluoromethylcyclohexane, perfluorocycloheptane, sulfur hexafluoride and mixtures thereof.

10. A contrast agent according to claim 7 wherein hyperpolarized gas is selected from the group consisting of hyperpolarized helium, hyperpolarized xenon, hyperpolarized neon and mixtures thereof.

11. A contrast agent according to claim 1 wherein said film-forming surfactant comprises a phospholipid.

12. A contrast agent according to claim 11 wherein said phospholipid is selected from the group consisting of a saturated phospholipid, a synthetic non-saturated phospholipid and mixtures thereof.

13. A contrast agent according to claim 12, wherein the saturated phospholipid is selected from the group consisting of dilauryloyl-phosphatidylcholine (DLPC), dimyristoylphosphatidylcholine (DMPC), dipalmitoyl-phosphatidylcholine (DPPC), diarachidoylphosphatidylcholine (DAPC), distearoyl-phosphatidylcholine (DSPC), dioleoylphosphatidylycholine (DOPC), 1,2 Distearoyl-sn-glycero-3-Ethylphosphocholine (Ethyl-DSPC), dipentadecanoylphosphatidylcholine (DPDPC), 1-myristoyl-2-palmitoylphosphatidylcholine (MPPC), 1-palmitoyl-2-myristoylphosphatidylcholine (PMPC), 1-palmitoyl-2-stearoylphosphatid-ylcholine (PSPC), 1-stearoyl-2-palmitoyl-phosphatidylcholine (SPPC),), 1-palmitoyl-2-oleylphosphatid-ylcholine (POPC), 1-oleyl-2-palmitoyl-phosphatidylcholine (OPPC), dilauryloyl-phosphatidylglycerol (DLPG) and its alkali metal salts, diarachidoylphosphatidylglycerol (DAPG) and its alkali metal salts, dimyristoylphosphatidylglycerol (DMPG) and its alkali metal salts, dipalmitoyl-phosphatidylglycerol (“DPPG”) and its alkali metal salts, distearolyphosphatidylglycerol (DSPG) and its alkali metal salts, dioleoylphosphatidylglycerol (DOPG) and its alkali metal salts, dimyristoyl phosphatidic acid (DMPA) and its alkali metal salts, dipalmitoyl phosphatidic acid “DPPA) and its alkali metal salts, distearoyl phosphatidic acid (DSPA), diarachidoyl phosphatidic acid (DAPA) and its alkali metal salts, dimyristoyl phosphatidyl-ethanolamine (DMPE), dipalmitoyl phosphatidylethanolamine (DPPE), DPPE-PEG, distearoyl phosphatidyl-ethanolamine (DSPE), DSPE-PEG, dioleylphosphatidylethanolamine (DOPE), diarachidoylphosphatidylethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), distearoyl sphingomyelin (DSSP) and mixtures thereof.

14. A contrast agent according to claim 11, wherein said dried material further comprises viscosity enhancers or stabilizers selected from linear and cross-linked poly- and oligo-saccharides, sugars, hydrophilic polymers like polyoxypropylene glycol and polyoxyethylene glycol.

15. The contrast agent of claim 11, wherein said dried material further comprises up to 50% by weight of a surfactants selected from fatty acids, esters and ethers of fatty acids and alcohols with polyols.

16. A composition for preparing a contrast agent in the form of an aqueous suspension containing gas-filled microvesicles useful in diagnostic imaging, said composition comprising: a) a dried material comprising at least one film-forming surfactant and b) a gas in contact with said dried material, said composition being reconstitutable upon contact with a physiologically acceptable aqueous carrier to form said gas-filled microvesicles suspension, wherein said gas has a pressure lower than atmospheric pressure.

17. A composition according to claim 16 wherein the dried material is a lyophilized material.

18. A composition according to claim 16 wherein the reduced pressure of said gas introduced into said container comprises between about 100 and about 900 mbar.

19. A composition according to claim 16 wherein the reduced pressure of said gas introduced into said container comprises between about 300 and about 700 mbar.

20. A composition according to claim 16 wherein the reduced pressure of said gas introduced into said container comprises between about 400 and about 600 mbar.

21. A composition according to claim 16, wherein the gas is selected from the group consisting of halogenated hydrocarbon gases, air, nitrogen, carbon dioxide, helium, krypton, xenon, argon, methane, radioactive gases, hyperpolarized gases and mixtures thereof.

22. A composition according to claim 21, wherein said film-forming surfactant comprises a saturated phospholipid or a synthetic non-saturated phospholipid or mixtures thereof.

23. A composition according to claim 22, wherein the saturated phospholipid is selected from the group consisting of dilauryloyl-phosphatidylcholine (DLPC), dimyristoylphosphatidylcholine (DMPC), dipalmitoyl-phosphatidylcholine (DPPC), diarachidoylphosphatidylcholine (DAPC), distearoyl-phosphatidylcholine (DSPC), dioleoylphosphatidylycholine (DOPC), 1,2 Distearoyl-sn-glycero-3-Ethylphosphocholine (Ethyl-DSPC), dipentadecanoylphosphatidylcholine (DPDPC), 1-myristoyl-2-palmitoylphosphatidylcholine (MPPC), 1-palmitoyl-2-myristoylphosphatidylcholine (PMPC), 1-palmitoyl-2-stearoylphosphatid-ylcholine (PSPC), 1-stearoyl-2-palmitoyl-phosphatidylcholine (SPPC),), 1-palmitoyl-2-oleylphosphatid-ylcholine (POPC), 1-oleyl-2-palmitoyl-phosphatidylcholine (OPPC), dilauryloyl-phosphatidylglycerol (DLPG) and its alkali metal salts, diarachidoylphosphatidylglycerol (DAPG) and its alkali metal salts, dimyristoylphosphatidylglycerol (DMPG) and its alkali metal salts, dipalmitoyl-phosphatidylglycerol (“DPPG”) and its alkali metal salts, distearolyphosphatidylglycerol (DSPG) and its alkali metal salts, dioleoylphosphatidylglycerol (DOPG) and its alkali metal salts, dimyristoyl phosphatidic acid (DMPA) and its alkali metal salts, dipalmitoyl phosphatidic acid “DPPA) and its alkali metal salts, distearoyl phosphatidic acid (DSPA), diarachidoyl phosphatidic acid (DAPA) and its alkali metal salts, dimyristoyl phosphatidyl-ethanolamine (DMPE), dipalmitoyl phosphatidylethanolamine (DPPE), DPPE-PEG, distearoyl phosphatidyl-ethanolamine (DSPE), DSPE-PEG, dioleylphosphatidylethanolamine (DOPE), diarachidoylphosphatidylethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), distearoyl sphingomyelin (DSSP) and mixtures thereof.

24. A container for use in preparing a gas containing contrast agent for diagnostic imaging, said container comprising
a dried material comprising at least one film forming surfactant; and
a gas;
wherein said gas is present at a pressure lower than atmospheric pressure.

25. A container according to claim 24, wherein the gas is present at a pressure of from about 100 to about 900 mbar.

26. A container according to claim 24, wherein the gas or gas mixture is present at a pressure of from about 300 to about 700 mbar.

27. A container according to claim 24, wherein the gas or gas mixture is present at a pressure of from about 400 to about 600 mbar.

28. A container according to claim 24 wherein the dried material is a lyophilized material.

29. A container according to claim 24, wherein the gas is selected from the group consisting of halogenated hydrocarbon gases, air, nitrogen, carbon dioxide, helium, krypton, xenon, argon, methane, radioactive gases, hyperpolarized gases and mixtures thereof.

30. A container according to claim 24, wherein said film-forming surfactant comprises a saturated phospholipid or a synthetic non-saturated phospholipid or mixtures thereof.

31. A container according to claim 24, wherein the saturated phospholipid is selected from is selected from the group consisting of dilauryloyl-phosphatidylcholine (DLPC), dimyristoylphosphatidylcholine (DMPC), dipalmitoyl-phosphatidylcholine (DPPC), diarachidoylphosphatidylcholine (DAPC), distearoyl-phosphatidylcholine (DSPC), dioleoylphosphatidylycholine (DOPC), 1,2 Distearoyl-sn-glycero-3-Ethylphosphocholine (Ethyl-DSPC), dipentadecanoylphosphatidylcholine (DPDPC), 1-myristoyl-2-palmitoylphosphatidylcholine (MPPC), 1-palmitoyl-2-myristoylphosphatidylcholine (PMPC), 1-palmitoyl-2-stearoylphosphatid-ylcholine (PSPC), 1-stearoyl-2-palmitoyl-phosphatidylcholine (SPPC),), 1-palmitoyl-2-oleylphosphatid-ylcholine (POPC), 1-oleyl-2-palmitoyl-phosphatidylcholine (OPPC), dilauryloyl-phosphatidylglycerol (DLPG) and its alkali metal salts, diarachidoylphosphatidylglycerol (DAPG) and its alkali metal salts, dimyristoylphosphatidylglycerol (DMPG) and its alkali metal salts, dipalmitoyl-phosphatidylglycerol (“DPPG”) and its alkali metal salts, distearolyphosphatidylglycerol (DSPG) and its alkali metal salts, dioleoylphosphatidylglycerol (DOPG) and its alkali metal salts, dimyristoyl phosphatidic acid (DMPA) and its alkali metal salts, dipalmitoyl phosphatidic acid “DPPA) and its alkali metal salts, distearoyl phosphatidic acid (DSPA), diarachidoyl phosphatidic acid (DAPA) and its alkali metal salts, dimyristoyl phosphatidyl-ethanolamine (DMPE), dipalmitoyl phosphatidylethanolamine (DPPE), DPPE-PEG, distearoyl phosphatidyl-ethanolamine (DSPE), DSPE-PEG, dioleylphosphatidylethanolamine (DOPE), diarachidoylphosphatidylethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), distearoyl sphingomyelin (DSSP) and mixtures thereof.

32. A two component kit comprising, as the first component, a composition as claimed in any of the claims from 16 to 22 and, as the second component a physiologically acceptable carrier liquid which, when admixed with the first component, provides an injectable ultrasound or magnetic resonance contrast agent.

33. A process for the manufacture of a composition as claimed in any of the claims from 16 to 22 to claim 16 which comprises the steps of:
a) preparing a dried material comprising at least one film-forming surfactant into a container;
b) applying a vacuum to said container;
c) introducing a gas into said container, up to a pressure which is lower than atmospheric pressure;
d) effecting a sealing closure of said container, thus obtaining the dried material in contact with an atmosphere of said gas having a pressure lower than atmospheric pressure.

34. The process of claim 33 wherein the gas is introduced into said container up to a pressure of from about 100 to 900 mbar.

35. The process of claim 33 wherein the gas is introduced into said container up to a pressure of from about 300 to 700 mbar.

36. The process of claim 33 wherein the gas is introduced into said container up to a pressure of from about 400 to 600 mbar.

37. The process according to claim 33 wherein the gas is selected from the group consisting of halogenated hydrocarbon gases, air, nitrogen, carbon dioxide, helium, krypton, xenon, argon, methane, radioactive gases, hyperpolarized gases and mixtures thereof.

38. The process according to claim 33 wherein said film-forming surfactant comprises a saturated phospholipid or a synthetic non-saturated phospholipid or mixtures thereof.

39. The process of claim 28, wherein the saturated phospholipid is selected from phosphatidic acid, phosphatidylcholine, phosphatidyl-ethanolamine, phosphatidyl-serine, phosphatidylglycerol, phosphatidyl-inositol, cardiolipin, sphingomyelin and mixtures thereof.

40. The process of claim 23, wherein said lyophilised material further comprises viscosity enhancers or stabilizers selected from linear and cross-linked poly- and oligo-saccharides, sugars, hydrophilic polymers like polyoxypropylene glycol and polyoxyethylene glycol.

41. The process of claim 38, wherein said lyophilised material further comprises up to 50% by weight of a surfactant selected from fatty acids, esters and ethers of fatty acids and alcohols with polyols.

42. A method of diagnostic imaging which comprises administering to a subject a contrast-enhancing amount of a contrast agent according to claims 1 to 15 and imaging at least a part of said subject.

43. A method according to claim 42 wherein an ultrasound image of said subject is generated.

44. A method according to claim 42 wherein a MR image of said subject is generated.

45. A method according to claim 42 wherein a scintigraphic image of said subject is generated.

46. The method according to claims 42 to 45 wherein said subject is a vertebrate and said contrast agent is introduced into the vasculature or into a body cavity of said vertebrate.
